(19) United States
(12) Patent Application Publication
(10) Pub. No.: US 2011/0217765 A1
(43) Pub. Date: Sep. 8, 2011

(54) Device for a Liquid Lock for a Fermentation Receptacle for Biological Material

(76) Inventor: Tilmann Hettasch, Fetsund (NO)

(21) Appl. No.: 13/061,029

(22) PCT Filed: Aug. 28, 2009
(86) PCT No.: PCT/NO09/00304

§ 371 Date: May 18, 2011

(30) Foreign Application Priority Data
Aug. 29, 2008 (NO) 20083733

(51) Int. Cl. C12M 1/107

(52) U.S. Cl. 435/300.1

(57) ABSTRACT

A liquid lock is for a fermentation receptacle for production of biogas, the liquid lock being provided as an annular liquid filled vessel encircling an upper portion of the receptacle wall(s), an inner portion of a fabric being fastened to the receptacle, an edge portion of a receptacle cover encircling the inner fabric portion, and an outer fabric portion extending upwardly along the edge portion and fastened to the receptacle cover, and conduit connections are provided between an internal space in the receptacle and the liquid lock.

The invention relates to a liquid lock device in a fermentation receptacle for biological material, and more particularly an annular liquid filled vessel encircling an upper portion of the receptacle wall(s), an inner portion of a fabric being fastened to the receptacle, an edge portion of a receptacle cover encircling the inner fabric portion, and an outer fabric portion being guided up along the edge portion and fastened to the receptacle cover, and conduit connections are provided between an inner space in the receptacle and the liquid lock.

It is known that when biological materials such as manure from domestic animals, abattoir waste, etc., undergoes an anaerobic fermentation process, methane gas (CH₄) is produced. The process is also known from old landfills for waste. Other gases such as carbon dioxide (CO₂) and hydrogen sulphide (H₂S) are released together with the methane gas. This gas mixture is colloquially often called “biogas”. Methane may be used as energy source for a series of purposes, and methane gas is for obvious reasons used as fuel in a plant to provide heat to such as room and/or water heating. To keep the decomposition process and the decomposition products, especially the methane, under control, one will as a rule let the fermenting mass be kept in a an airtight receptacle to prevent access of oxygen from the outside to the mass and for the collection of the methane gas to be effective.

The decomposition is time-consuming such that the dung mass needs a certain dwell time in the tank, and it is an advantage to stir the mass, either by agitator or by circulation by means of a pump. The decomposition is thought to be most stable at a temperature of about 35-40° C. It is therefore important to avoid too large heat losses from the tank to the cold surroundings, which might require some form of insulation.

It is also known to let heat loss from the fermentation tank go mainly through the top of the tank to reduce or eliminate possible snow load on the roof structure.

In known plants for production of biogas a raw gas being produced during the fermentation process is lead out of the tank to units where water vapour, carbon dioxide and hydrogen sulphide are separated before the biogas is carried to such, for example, a combustion plant.

Prior art exhibits in part complicated solutions for gas tight joining of receptacle walls and cover. The challenges are among other things to be able to provide the possibility of quick and simple dismantling of the receptacle cover for inspection, cleaning etc.

The object of the invention is to remedy or reduce at least one of the prior art drawbacks.

The object is achieved by features stated in the below description and in the following claims.

The invention provides a simplified liquid lock for use as a safety valve in a fermentation receptacle for recovery of biogas by anaerobic fermentation of biological material. The liquid lock is provided in that a portion of a fabric forms an annular vessel encircling an upper portion of the receptacle. An edge portion of the receptacle cover is submerged in the liquid filled vessel and forms fluid conduits from the inside of the receptacle and out into the liquid lock. The fabric may be a bottom fabric forming an internal, fluid sealing barrier in the receptacle, as an edge portion of the bottom fabric extends down along the outside of the receptacle side wall.

The invention relates more particularly to a device for a plant for recovery of biogas from biological material by anaerobic fermentation, wherein a fermentation mass is contained in a receptacle, and a receptacle cover is releasably interconnected with side wall(s) of the receptacle forming a closed space over the fermenting mass, and at least one fluid communicating conduit forming a connection between the space and the surroundings, wherein the at least one fluid communicating conduit is provided with a first valve arranged to be able to limit the gas pressure over the fermentation mass to a first gas pressure, as the first valve is formed by the at least one conduit being submerged in an annular, liquid filled vessel, characterised in that
an inner portion of a fabric encircles an upper portion of the receptacle;
an edge portion of the receptacle cover encircles the inner portion of the fabric;
an outer portion of the fabric encircled the edge portion of the receptacle cover;
the fabric extending continuously down along the outside wall surface of the receptacle wall from the upper edge of the receptacle and up along the edge portion of the receptacle cover, and the outer portion of the fabric being fastened to a portion of the receptacle cover above the edge portion.

The fabric may be a bottom fabric forming a fluid sealing barrier in the receptacle.

The inner and outer portions together may form an edge portion of the bottom fabric.

The fabric and the receptacle cover may be releasably fastened to the receptacle by a clamping ring being provided internally in the vessel and being arranged to be able to clamp a portion of the fabric and the receptacle cover against a fabric abutment provided externally on the receptacle wall.

The edge portion of the receptacle cover may be provided with at least one gas drainage opening arranged to be able to be submerged in the vessel.

The at least one gas drainage opening may be formed as a perforation in the receptacle cover.

The fabric may be provided with fasteners arranged for releasable fastening of the outer portion to the receptacle cover.

The outer portion of the fabric may be covered with a heat insulating material.

In the following is described an example of a preferred embodiment which is illustrated in the accompanying drawings, where:

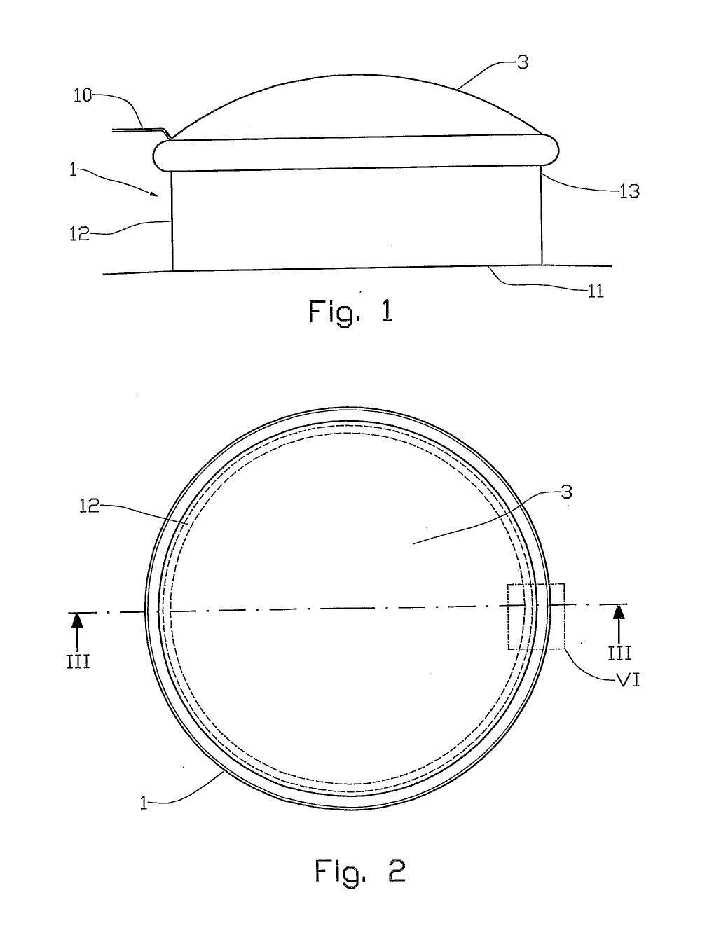

FIG. 1 shows a side view of a receptacle for a plant of the invention;

FIG. 2 shows a plan view of the receptacle of FIG. 1;

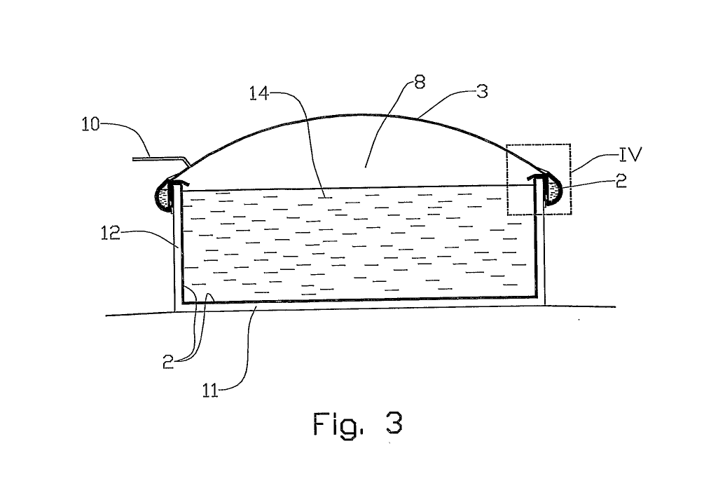

FIG. 3 shows a cross-section through the receptacle of FIG. 2; and

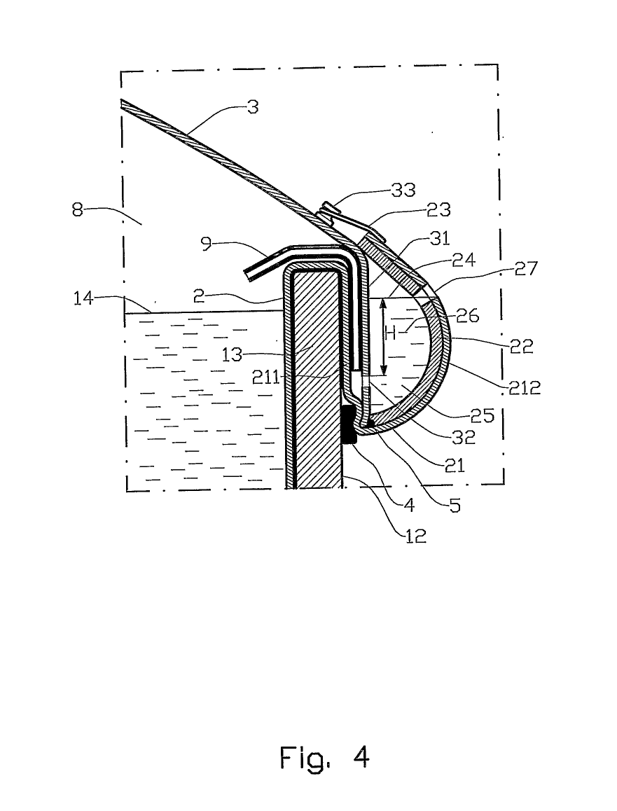

FIG. 4 shows in greater detail a section IV of the receptacle of FIG. 3.

A plant according to the invention comprises at least one cylindrically shaped receptacle 1. The receptacle is in a per se known way provided with a bottom 11, which is interconnected with a receptacle wall 12. A bottom fabric 2 formed of a fluid tight, flexible material provides an internal sealing barrier in the receptacle 1 as it extends continuously over the whole of the receptacle bottom 11 and up along the receptacle wall 12, and an edge portion 21 extends over the receptacle wall 12 and down along the external wall surface of an upper portion 13 of the receptacle 1. The bottom fabric 2 makes thereby possible that the receptacle 1 floor 11 and sidewall 12 may be formed of materials, which first of all provides support for the bottom fabric 2.

The receptacle 1 is arranged for containment of biological material 14 such as domestic animal manure, which on anaerobic fermentation gives off methane gas among other things.

A fabric abutment 4 encircles the receptacle 1 at a distance from the upper edge of the receptacle wall 12. The fabric abutment 4 is formed from a material arranged to take up compression forces from a locking ring 5.

A receptacle cover 3 extends over the whole of the receptacle 1 base area as an edge portion 31 is lead down along an upper portion 13 of the receptacle 1, on the outside of an inner zone 211 of the bottom fabric 2 edge portion 21 to abutment against the fabric abutment 4. The receptacle cover 3 thus forms a closed space 8 over the biological material 14 contained in the receptacle 1. In the receptacle cover 3 edge portion 31 there is provided several perforations forming openings 32 for draining gas from the enclosed space 8. The openings 32 are arranged between the fabric abutment 4 and the upper edge of the receptacle wall 12. The receptacle cover 3 is dome shaped.

Between the inner edge zone 211 of the bottom fabric 2 and the externally situated edge portion 31 of the receptacle cover 3 there is provided a series of conduits 9 connecting the enclosed receptacle 1 space 8 with the openings 32. The conduits 9 are shown here as hose connections, but the connecting conduits may obviously be provided in a large number of ways, for example by there being provided distance keeping elements (not shown) between the bottom fabric 2 and the receptacle cover 3 such that several interrupted annuli are provided.

The clamp ring 5 abuts the edge portions 211, 31 of the fabrics 2, 3, and the fabric abutment 4 and provides thereby a releasable connection between the fabrics 2, 3 and the receptacle wall 12.

An outer edge zone 212 of the bottom fabric 2 extends from the clamp ring 5 abutment surface against the bottom fabric 2 and the bottom fabric 2 periphery. The outer edge zone 212 is provided with a heat-insulating layer 22.

The periphery of the bottom fabric is provided with a series of fasteners 23 arranged for fastening in corresponding fastening means 33 provided on the topside of the receptacle cover 3 and close to the receptacle cover 3 abutment against the upper edge of the receptacle wall 12.

The outer edge zone 212 of the bottom fabric 2 which is provided with the heat insulating layer 22, is continued upward outside the clamp ring 5 and the receptacle cover 3 edge portion 31 and is fastened to the receptacle cover 3, as the bottom fabric 2 fasteners 23 are fastened to the receptacle cover 3 fastening means 33. The edge portion 21 of the bottom fabric 2 thus forms an annular vessel 24 which may be filled with a liquid 25 and forming a liquid lock 26.

The vessel 24 is provided with at least one liquid drain opening 27 formed as a perforation through the bottom fabric 2 in a suitable vertical distance from the openings 32 in the receptacle cover 3 to provide a prescribed depth of liquid in the vessel 24. The ring shaped vessel 24 is at the assembly stage filled with liquid 26 from a suitable source (not shown) such as from the local water supply. The vessel 24 will catch rain and melt water running off the receptacle cover 3 surface. If the supply of liquid 26 is less than the natural evaporation, the vessel must be replenished from said source.

The openings 32 in the receptacle cover 3 for draining gas from the enclosed space 8 are submerged in the liquid lock 25 to a depth H providing a desired pressure under the receptacle cover 3. The pressure is typically 5-30 millibars, and this is by experience sufficient to keep the receptacle cover 3 inflated when it is subjected to extra loads in the form of wind or snow.

A gas conduit 10 extends from the space 8 over the biological material 14 to devices (not shown) for storage and use of the methane gas.

If the pressure in the space 8 exceeds the defined limit value P, gas will flow out through the conduits 9 and the openings 32 and through the liquid lock 26 and out to the surroundings, the gas passing between the bottom fabric 2 and the receptacle cover 3 at the fasteners 23, 33 or through liquid drain openings 27, as the liquid lock functions as a safety valve for the gas filled space 8 of the receptacle 1.

The liquid 26 content in the vessel 24 provides a load at the fastening of the bottom fabric 2 to the receptacle cover 3 which reduces the loading that the receptacle cover 3 applies on the clamping connection between the clamp ring 5, the edge portion 31 of the receptacle cover 3 and the fabric abutment 4 due to the internal gas pressure in the receptacle 1. By increasing the volume of the vessel 24, a higher pressure might be utilised in the receptacle 1, assuming that other relevant variables such as the strength of the bottom fabric 2 and the receptacle cover 3 and the liquid depth H is increased.

1. Device for a plant for recovery of biogas from biological material by anaerobe fermentation, wherein a fermentation mass is contained in a receptacle, and a receptacle cover is releasably interconnected with side wall(s) of the receptacle forming a closed space over the fermentation mass, and at least one fluid communication conduit forming a connection between the space and the surroundings, wherein the at least one fluid communication conduit is provided with a first valve arranged to be able to limit the gas pressure over the fermentation mass to a first gas pressure, as the first valve is formed by that the at least one conduit being submerged in an annular, liquid filled vessel, wherein:
an inner portion of a fabric encircles an upper protion of the receptacle;
an edge portion of the receptacle cover encircles the inner portion of the fabric;
an outer portion of the fabric encircles the edge portion of the receptacle cover;
the fabric extending downward along the exterior wall surface of the receptacle wall from the upper edge of the receptacle and upward along the receptacle cover edge portion, and the outer portion of the fabric being fastened to a portion of the receptacle cover at or above the edge portion.

2. Device according to claim 1, wherein the fabric is a bottom fabric forming a fluid sealing barrier in the receptacle.

3. Device according to claim 2, wherein the inner and outer portions together form an edge portion of the bottom fabric.

4. Device according to claim 1, wherein the fabric and the receptacle cover are releasably fastened to the receptacle by a clamp ring being provided internally in the vessel and being arranged to be able to clamp a portion of the fabric and the receptacle cover against a fabric abutment provided externally on the receptacle wall.

5. Device according to claim 1, wherein the edge portion of the receptacle cover is provided with at least one gas drainage opening arranged to be able to be submerged in the vessel.

6. Device according to claim 5, wherein the at least one gas drainage opening is formed as a perforation in the receptacle cover.

7. Device according to claim 1, wherein the fabric is provided with fastening devices arranged for releasable fastening of the outer portion to the receptacle cover.

8. Device according to claim 1, wherein the outer portion of the fabric is covered with a heat insulating material.
